SEQUENCE LISTING (2 sequences)

SEQ ID NO: 1 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

(19) United States
(12) Patent Application Publication
(10) Pub. No.: US 2021/0393688 A1
(43) Pub. Date: Dec. 23, 2021

(54) MODIFIED PDC LINE FOR SECRETING A CYTOKINE

(71) Applicants: PDC LINE PHARMA, La Tronche (FR); ETABLISSEMENT FRANCAIS DU SANG, La Plaine Saint-Denis (FR)

(72) Inventors: Dalil HANNANI, Grenoble (FR); Joel PLUMAS, Grenoble (FR)

(21) Appl. No.: 17/288,019

(22) PCT Filed: Oct. 23, 2019
(86) PCT No.: PCT/EP2019/078845

§ 371 Date: Apr. 23, 2021

(30) Foreign Application Priority Data
Oct. 23, 2018 (FR) 1859773

(51) Int. Cl. A61K 35/17; A61K 39/12; A61K 39/00; A61K 38/20; C12N 5/0784

(52) U.S. Cl. CPC A61K 35/17; A61K 39/12; A61K 39/0011; A61K 38/2086; C12N 2502/11; C12N 5/0639; A61K 2039/5156; C12N 2510/00; A61K 38/2013

(57) ABSTRACT

The present invention relates to a genetically modified PDC (plasmacytoid dendritic cell) line for secreting a cytokine and to its use for increasing the expansion of antigen-specific cells in immunotherapies.

### FIELD OF THE INVENTION

The present invention relates to a plasmacytoid dendritic cell (PDC) line genetically modified for secreting a cytokine, and its use to increase the expansion of antigen-specific cells in immunotherapies.

### STATE OF THE ART

Immunotherapy approaches seeking to promote the production of antigen-specific cells are known. An approach using a plasmacytoid dendritic cell (PDC) line (called “PDC*line”) to induce antigen-specific cytotoxic cells from donor mononuclear cells (MNCs), in particular peripheral blood mononuclear cells (PBMCs), has been developed by the inventors (WO 2009/138489). This approach consists in culturing MNCs in the presence of irradiated and antigen-loaded “PDC*line”, the mononuclear cells sharing at least one HLA molecule with “PDC*line” (HLA-A2, for example).

Work conducted by the applicants has shown the attractiveness of this therapeutic approach and its considerable potential in the immunotherapy treatment of cancers such as melanoma or lung cancers (Aspord et al., 2012, unpublished data).

The ability to induce the expansion of antigen-specific cells is a determining factor in the implementation of these new therapeutic approaches. It is known, in particular from the application WO 2009/138489, that the presence of cytokines is necessary for this expansion. The experimental protocol described consists, the first week, in a co-culture of irradiated and antigen-loaded PDC*line with MNCs performed without cytokine, then, the second week, in a new stimulation with irradiated antigen-loaded PDC*line and IL-2.

The importance of cytokines in the development mechanisms of the various immune responses is well known to the person skilled in the art, in particular for IL-2 and IL15 (Waldmann, Nat Rev 2006). IL-2 is produced mainly by activated T cells, whereas IL-15 and its associated receptor IL-15Ra (Dubois et al., 2002), is produced by monocytes or myeloid dendritic cells (MDCs) (Jakobisiak et al., 2011). Transient or permanent modification of in vitro-generated MDCs to produce IL15 has been described for the purpose of enhancing cell function (van den Bergh et al., 2015, Steel et al (2010) or Zhang et al. (2014)) in the induction of antitumor response (NK, antibody or antigen-specific T).

Even if MDCs and PDCs are both antigen-presenting cells, these are two different cell types capable of inducing different types of immune responses depending on the origin of the pathogen or antigen and the environmental context in general, differentiated by a number of characteristics: origin, tissue localization, Toll-like receptor expression, cytokine production, among others. None of these papers on MDCs modified to express IL15 mention the possibility of using PDCs, or even consider an at least equivalent effect on PDCs with IL15 or even IL2.

While the conventional activation and expansion method described in WO 2009/138489 already achieves particularly high levels of expansion (Aspord et al., 2010), it remains advantageous to improve this new therapeutic approach at the level of antigen-specific cell expansion.

### DISCLOSURE OF THE INVENTION

The invention thus relates to a new PDC line genetically modified to express a cytokine selected from interleukin 15 (IL15) and interleukin 2 (IL2).

In particular, the genetically modified PDC cells according to the invention are loaded with one or more antigens, in peptide form.

The invention also relates to the use of the new genetically modified PDC line to amplify antigen-specific cells, in particular mononuclear cells (MNCs), more particularly peripheral blood mononuclear cells (PBMCs).

The invention also relates to a combination product or kit, comprising on the one hand a genetically modified PDC line according to the invention and on the other hand mononuclear cells (MNCs), in particular for simultaneous use in the treatment of diseases by immunotherapy.

The invention also relates to a vaccine composition comprising a genetically modified PDC line according to the invention and a suitable vehicle for its administration.

### DESCRIPTION OF THE FIGURES

FIG. 1 represents the measurement of the expression of the CD34 molecule on the non-transduced PDC*line (left) or transduced with retroviral supernatant encoding Il-2 or IL-15.

FIG. 2 represents the expression of IL2 or IL15 by the PDC*line transduced with IL2 or IL15 retroviral supernatant. Cytokines are detected at the level of messenger RNA (A, normalized to G6PDH) or in the supernatant of transduced cells (B, in pg/ml).

FIG. 3 represents the expansion of anti-Melan-A CD8+ T cells after 14-day co-culture of the PDC line, unmodified or genetically modified with IL2 or IL15, loaded with Melan-A antigen, and mononuclear cells from 3 healthy donors. Anti-Melan-A CD8+ T cells are detected by flow cytometry using HLA-A2/Melan-A multimers. In A, a representative experiment is shown; in B, the results of 3 experiments are represented (% CD8+ multimer cells).

FIG. 4 represents the function of CD8+ cells from the 14-day co-culture. The function of cytotoxic cells is objectified by the detection of intracytoplasmic IFN_{γ} after stimulation by the target line (T2) loaded with the Melan-A derived peptide used. In A, the illustration of the intracytoplasmic detection of the cytokine on all specific and non-specific CD8+ cells obtained with the different lines. In B, the percentage of IFN_{γ}-positive CD8+ cells in non-specific (Multimer-) and specific (Multimer+) cells generated with each line.

### DETAILED DESCRIPTION OF THE INVENTION

The invention thus relates to a new PDC line genetically modified to express a cytokine selected from IL15 and IL2.

PDC lines useful according to the invention for being genetically modified, also called initial PDC lines, are well known to the skilled person, in particular described in EP 1 572 989 and WO 2009/138489. In particular, these are cells obtained from PDC leukemia cells. Particular mention may be made of the GEN2.2 and GEN3 lines deposited under numbers 2938 and 3110 at the CNCM (Collection Nationale de Cultures de Microorganismes, Institut Pasteur, 25 rue du Docteur Roux, 75015 Paris) and of all lines derived from PDC leukemia cells.

“Lines derived from PDC leukemia cells” means lines derived from tumor nodules following the injection of PDC leukemia cells into immunodeficient mice, these nodules being dilacerated to obtain a cell suspension which is cultured in a synthetic medium promoting the growth of said line.

“Expression of a cytokine selected from IL15 and IL2” means, according to the invention, the secretion of the cytokine by the genetically modified line.

The sequences of the cytokines are well known to the skilled person. Particular mention may be made, for IL2, of the protein sequence identified under the UniProtKB entry P60568 and the coding sequence identified under the Ensembl entry ENSG00000109471 and, for IL-15, the protein sequence identified under the UniProtKB entry P40933 and the coding sequence identified under the Ensembl entry ENSG00000164136. The variants or fragments of these cytokines which have the same activity as the above sequences are also part of the invention. Preferably the cytokines are the human cytokines identified above.

“Genetically modified PDC line” means, according to the invention, any line transduced with a viral particle allowing integration of the gene into the genome of the line, which virus may be an adenovirus, a lentivirus or a retrovirus. Preferably a retrovirus of the Moloney murine leukemia virus (Mo-MLV) type is used. These retroviral particles, being produced by the HEK 293T encapsulation line, express naturally or after transfection the retroviral gag/pol and env sequences.

The person skilled in the art will know how to prepare a viral particle comprising a coding sequence for the gene(s) of interest and the regulatory elements, in particular the sequences used to allow the expression of cytokines in genetically modified mammalian cells. Particular mention may be made of the viral envelope sequences, allowing the entry of the virus into the cell, such as 4070A, MK-G, HA, LCMV, RD114, HIV, VSV, MLV, GALV, BAEV), the promoter sequences allowing the initiation of the transcription of the gene of interest, such as long terminal repeat (LTR) sequences, modified or not, PGK, EF-1, CMV, SFFV, RSV, or SV40, sequences enhancing transduction efficiency such as cPPT, sequences facilitating or initiating translation, such as the internal ribosome entry site (IRES) sequence (Pelletier, Nature 1988), or the Kozak sequence (Kozak, Nucl acid Res 1991), sequences promoting protein expression, such as the WPRE sequence (Donello et al., J. Virol 1998). Preferably the LTR, IRES, Kozak and WPRE sequences will be used.

Methods for transducing PDC cells with a viral particle to obtain a new PDC line genetically modified to express a cytokine selected from IL15 and IL2 are also known to the skilled person. Particular mention may be made of the use of polycationic agents, such as polybrene or DOGS (dioctadecylamidoglycylspermine), or of agents promoting contacts between virus and cells of interest, such as fibronectin fragments (RetroNectin). Preferably the method using RetroNectin is used.

Preferably, the cytokine alone is expressed. In certain cases, in particular for IL15, the PDC line also expresses the cytokine receptor, in particular the IL15Ra receptor. The IL15Ra receptor is well known to the skilled person, in particular its protein sequence.

In the case where the PDC line modified according to the invention does not express said receptor, the PDC line may also be modified to express said receptor, according to the usual methods of the art.

The modified PDCs according to the invention are particularly useful for the amplification of antigen-specific cells, in particular mononuclear cells (MNCs), more particularly peripheral blood mononuclear cells (PBMCs).

Although they can be used directly, the modified PDC cells according to the invention are preferably associated with antigens, in particular peptide antigens.

In the context of the present invention, the term “antigen” defines a molecule or part of a molecule recognized by cells of the immune system and capable of triggering a cell-mediated immune reaction. The antigens according to the present invention may be native or modified, tumor or microbial (in particular bacterial or viral) antigens, such as peptides, proteins, glycopeptides, glycoproteins, phosphorylated proteins.

The skilled person will choose the antigen(s) to be associated with the modified PDC line according to the disease to be treated.

In a preferred implementation of the invention, the antigens are peptides obtainable from antigenic proteins of tumor or viral origin. These peptides are well known to the skilled person, described in particular in numerous patent applications or patents, in particular EP 1 485 719, EP 2 113 253, U.S. Pat. Nos. 7,087,712, 7,528,224, WO 94/020127, WO 95/02553, WO 98/22589, WO 2000/003693, WO 2000/020445, WO 2000/052163, WO 2000/078806, WO 2004/067023, WO 2007/036638, WO 2007/039192, WO 2008/045286, WO 2011/012720, WO 2015/0965572 and WO 2016/179573.

A number of tumor antigens that may be used are also described in databases available online, such as for example at the addresses http://cvc.dfci.harvard.edu/tadb/ or https://docs.google.com/spreadsheets/u/1/d/1BFn5_mu7ogUh35NsLUozj9EB2rbLtj7XjMyt7BoYNxg/pubhtml?widget=true&headers=false#gid=1609210712 or in publications such as Djureinovic et al. (JCI Insight. 2016; 1(10):e86837).

Similarly, a number of viral antigens that may be used are also described in databases available online, such as for example at the address https://www.iedb.org/ or http://crdd.osdd.net/raghava/antigendb/.

According to a particular embodiment of the invention, the peptides obtainable from tumor antigens can be selected from the peptides included in the sequence of the antigens CEA, NY-BR1, Her-2/Neu, PSA, RAGE-1, PRAME, TRP-2, MAGE-A1, MAGE-A2, MAGE-A3, MAGE-A4, MAGE-A9, MAGE-A10, MAGE-C1, MAGE-C2, Multi-MAGE, MUC-1, p53, hTERT, survivin, melan-A/MART-1, GP100, tyrosinase, CAMEL or NY-ESO1, modified or not, alone or in combination. Similarly, any antigen selected from among mutated proteins or proteins resulting from the transcription of a messenger RNA generated by a new reading frame of a nucleic sequence (neo-antigen).

According to another embodiment of the invention, the peptides obtainable from viral antigens can be selected from the peptides included in the sequence of the antigens env, nef, gp41, gp120, gag or pol of the HIV virus, HBc or HBs of the HBV virus, core, NS3 or NS5 of the HCV virus, Flu M1 of the influenza virus, CMVpp65 of the CMV virus, BMLF1, LMP2, EBNA-2 or EBNA-3a of the EBV virus, LTA or VOP1 of the BKV virus, the nucleoprotein of the Hatan virus, NS3 of the Dengue virus, E6 and E7 of the HPV virus, protein E, NS3, or BS4b of the West Nile virus, modified or not, alone or in combination.

Preferential mention may be made of Melan-A, gp100, Tyrosinase, MAGE-A3, MAGE-A4, MAGE-A10, Multi-MAGE, CTAG2, CTAG1, Survivin, Her-2/Neu, hTERT and MUC1.

According to a first preferred embodiment of the invention, the genetically modified PDC cells are loaded with one or more antigens. They will also be said to be pulsed, i.e., incubated with one or more antigens.

The invention thus relates to a genetically modified PDC line as defined above and in the examples, loaded with one of the antigens listed above.

According to another embodiment of the invention, the cells are also genetically modified to express said antigens. The genetic modification tools are the same as those used to modify PDC cells to express cytokines. In the case of antigens, the skilled person will also choose genetic elements that allow the antigens to be expressed at the cellular level so that they are transformed into epitopes and presented by HLA molecules, in vitro or in vivo. Such elements are also well known to the skilled person (Hu, Immunological review 2011).

For therapeutic use, the genetically modified PDC lines according to the invention, as defined above and below, are irradiated lines.

Irradiation methods and conditions for inhibiting the growth and multiplication capabilities of the transformed PDC lines according to the invention are well known to the skilled person, in particular described in WO 2009/138489.

The invention also relates to the use of the new genetically modified PDC line to amplify antigen-specific cells, in particular mononuclear cells (MNCs), more particularly peripheral blood mononuclear cells (PBMCs).

The invention also relates to the new genetically modified PDC line for use in therapy, in particular for the treatment of diseases by immunotherapy.

The invention also relates to a combination product or kit, comprising on the one hand a genetically modified PDC line according to the invention and on the other hand mononuclear cells (MNCs), in particular for simultaneous use in the treatment of diseases by immunotherapy.

The disease treated will depend essentially on the antigens that are associated with the line according to the invention, with for example tumor antigens being used for the treatment of cancers and viral antigens for the treatment of viral infections.

The invention also relates to a vaccine composition comprising a genetically modified PDC line according to the invention and a suitable vehicle for its administration.

The invention also relates to a method for preventing and/or treating cancers and/or infectious diseases, characterized in that an irradiated and pulsed genetically modified PDC line according to the invention is injected into a patient in need of the treatment, the antigen-specific cells of said patient and the PDCs sharing at least one major histocompatibility complex (MHC) allele.

The invention also relates to a method for preventing and/or treating cancers and/or infectious diseases, characterized in that specific effectors obtained by incubating a PDC line according to the invention with at least one antigen are injected into a patient in need of the treatment, the pulsed PDCs, possibly irradiated, then being brought into contact with antigen-specific cells of said patient and cultured, the PDCs and the antigen-specific cells sharing at least one major histocompatibility complex (MHC) allele.

“Specific effector” means, according to the invention, immune cells capable of recognizing a specific antigen or a product derived from this antigen, in particular cytotoxic effectors and more particularly T cells specific to the antigen used, and in particular CD8+.

These methods are described in particular in WO 2009/138489.

### Examples

### Example 1: Generation of PDC*Line Lines Genetically Modified to Express IL-2 or IL15

The gene sequences of interest encoding IL2 (SEQ ID NO 1) and IL15 (SEQ ID NO 2) were synthesized by ThermoFischer (https://www.thermofisher.com/fr/fr/home/life-science/cloning/gene-synthesis/geneart-gene-synthesis.html) and provided in a plasmid allowing plasmid amplification after transformation of DH5alpha bacteria (NEB). The sequence of interest (IL2 or IL15) was then subcloned into plasmid SFGΔCD34 (Quintarelli, Blood 2007, generously provided by Dr. M. Pule) between the Sal-I and Mul-I restriction sites using Gibson Assembly technology (NEB). The presence of a truncated CD34 sequence in the SFGΔCD34 plasmid (without an intracytoplasmic domain) allows selection of cells expressing the gene of interest. A retroviral suspension was then obtained by triple transfection of the HEK-293T line with the SFGΔCD34-IL2 or SFGΔCD34-IL15 plasmid and the MoMLV gag-pol pEQ-PAM3(-E) and RD114 env expression plasmids (generously provided by Dr. M. Pule and Dr. M. Collins (Cosset, J Virol 1995)) in the presence of GeneJuice (VWR).

Cells of the PDC line were subsequently transduced with the retroviral supernatant corresponding to IL2 or IL15 in the presence of RetroNectin (Takara) and CD34 expression was measured, reflecting the transduction efficiency. This line was derived from cells of a patient with PDC leukemia (Chaperot, Blood 2001), the patient from which the GEN2.2 and

GEN3 lines were derived.

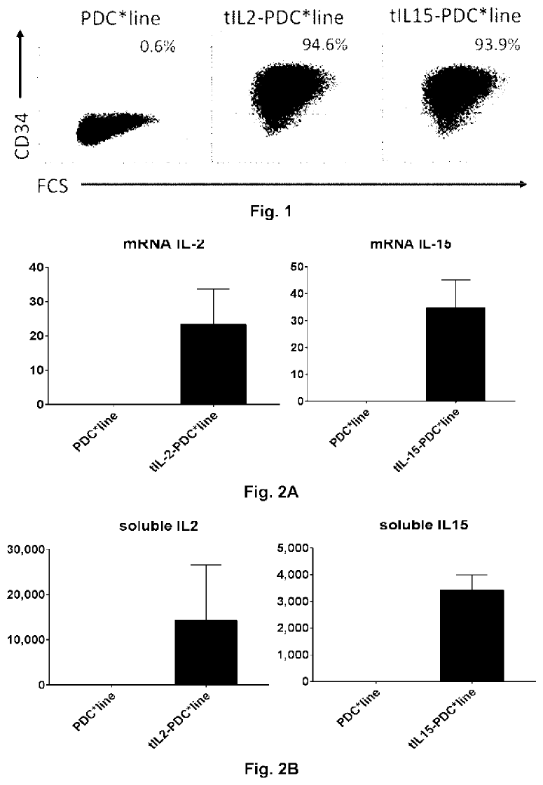

As shown in FIG. 1, more than 90% of PDC*line was transduced.

This CD34 expression is correlated with the expression of IL2 or IL15 genes. Indeed, RT-qPCR analysis (TaqMan technique, Roche) shows a high relative expression, compared with the G6PDH gene, for IL2 or IL15, by a factor greater than 20 or 30, respectively (FIG. 2A). Furthermore, using the CBA (BD) or ELISA (R&D) technique, both cytokines are found produced in soluble form in the supernatant of the corresponding genetically modified lines at the concentration of 20 000 pg/ml and 2 500 pg/ml for IL2 and IL15, respectively (FIG. 2B).

### Example 2: Expansion of CD8+ Lymphocytes after Co-Culture of MNCs with Transduced Lines Loaded with a Tumor Peptide

The ability of the modified lines was then assessed using co-culture with HLA-A2+ healthy donor mononuclear cells (MNCs). Briefly, cells from the genetically modified and unmodified PDC line were loaded with Melan-A_{26L-35} peptide, irradiated, and then cultured with MNCs in 24-well plates for 14 days. Different amounts of PDC line cells were added to the 2 million MNCs (10/1 or 20/1) per well. At D7, the cells were restimulated with the Melan-A-loaded PDC line in the presence of soluble IL-2. At D14, the expansion of anti-Melan-A CD8+ lymphocytes is measured using fluorochrome-labeled Melan-A/A2 dextramer (Multimer) and anti-CD3 and CD8 antibodies (Beckman Coulter).

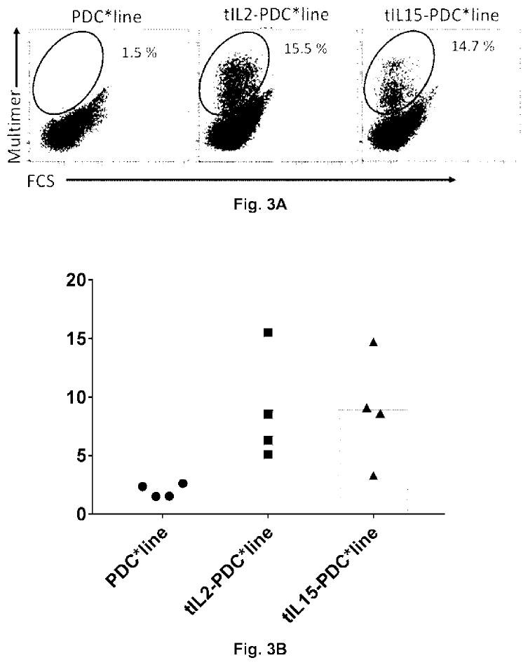

The results presented in FIG. 3 show that the expression of IL2 or IL15 by the PDC line allows a stronger expansion than the unmodified line. This increase is on average a factor greater than or equal to 3.7.

### Example 3: Functionality of Effectors Generated with the PDC Line Modified or not by IL2 or IL15: Intracytoplasmic IFNγ Expression

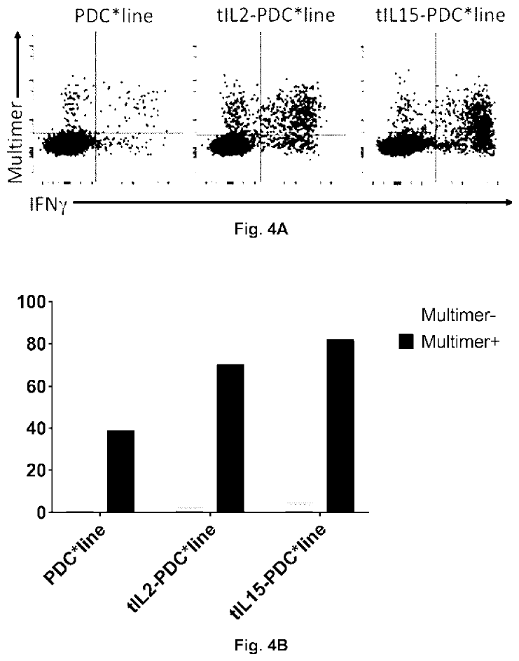

T cells amplified after 14-day co-culture in the presence of the Melan-A-loaded PDC line, transduced or not by IL-2 or IL15, were labeled with Melan-A/A2 dextramer (Multimer) and restimulated with the Melan-A-loaded T2 target line in the presence of a degranulation inhibitor, GolgiSTOP (Beckton Dickinson) for 4 h. The cells were then labeled with anti-IFN₁ antibody (Becton Dickinson) and anti-CD3 and CD8 antibodies. FIG. 4A represents the phenotyping obtained representing the information of IFNγ positive cells according to the multimer labeled cells. FIG. 4B shows that only Melan-A specific cells (multimer+) produce IFNγ when stimulated with the T2/Melan-A lineage. The results show that Melan-A-specific lymphocytes amplified in the presence of the PDC line modified to express IL2 or IL15 secrete 1.8 to 2.1 times more IFNγ than Melan-A-specific lymphocytes amplified with the unmodified line.

### REFERENCES

Aspord C, et al., Novel cancer vaccine strategy based on HLA-A*0201 matched allogeneic plasmacytoid dendritic cells. PLoS One. 2010 May 4; 5(5):e10458
Aspord et al., HLA-A(*)0201(+) plasmacytoid dendritic cells provide a cell-based immunotherapy for melanoma patients. J Invest Dermatol. 2012 October; 132(10):2395-406
Djureinovic et al., Profiling cancer testis antigens in non-small-cell lung cancer, JCI Insight. 2016; 1(10):e86837
Dubois et al., IL-15R Recycles and Presents IL-15 In trans to Neighboring Cells, Immunity, Vol. 17, 537-547, November, 2002,
Dubsky et al., IL-15-induced human DC efficiently prime melanoma-specific naive CD8+ T cells to differentiate into CTL, Eur. J. Immunol. 2007. 37: 1678-1690
Jakobisiak et al., Interleukin 15 as a promising candidate for tumor immunotherapy, Cytokine & Growth Factor Reviews 22 (2011) 99-108
Steel et al., Interleukin-15 and Its Receptor Augment Dendritic Cell Vaccination against the neu Oncogene through the Induction of Antibodies Partially Independent of CD4 Help, Cancer Res; 70(3) Feb. 1, 2010, 1072-1081
Vand den Bergh et al., Transpresentation of interleukin-15 by IL-15/IL-15Ra mRNA-engineered human dendritic cells boosts antitumoral natural killer cell activity, Oncotarget, 2015, Vol. 6, No. 42, 44123-44133
Zhang et al., Dendritic cell-derived interleukin-15 is crucial for therapeutic cancer vaccine potency, OncoImmunology 3:10, e959321; Nov. 1, 2014 EP 1 485 719, EP 2 113 253, U.S. Pat. Nos. 7,087,712, 7,528,224, WO 94/020127, WO 95/02553, WO 98/22589, WO 2000/003693, WO 2000/020445, WO 2000/052163, WO 2000/078806, WO 2004/067023, WO 2007/036638, WO 2007/039192, WO 2008/045286, WO 2009/13848, WO 2011/012720, WO 2015/0965572 and WO 2016/179573
http://cvc.dfci.harvard.edu/tadb/
https://docs.google.com/spreadsheets/u/1/d/1BFn5_mu7ogUh35NsLUozj9EB2rbLtj7XjMyt7BoYNxg/pubhtml?widget=true&headers=false#gid=1609210712

1. A plasmacytoid dendritic cell (PDC) line genetically modified to express a cytokine, wherein the cytokine is selected from interleukin 15 (IL15) and interleukin 2 (IL2).

2. The genetically modified PDC line according to claim 1, wherein the PDC line being modified is a PDC line obtained from leukemic plasmacytoid dendritic cells.

3. (canceled)

4. The genetically modified PDC line according to claim 1, wherein only the cytokine selected from interleukin 15 (IL15) and interleukin 2 (IL2) is expressed.

5. The genetically modified PDC line according to claim 1, wherein the genetically modified PDC line is loaded with one or more antigens.

6. The genetically modified PDC line according to claim 1, wherein the genetically modified PDC line is also genetically modified to express one or more antigens.

7. The genetically modified PDC line according to claim 5, wherein the antigens are peptide antigens.

8. The genetically modified PDC line according to claim 1, wherein the genetically modified PDC line is irradiated.

9.-10. (canceled)

11. A combination product or kit, comprising a genetically modified PDC line according to claim 1 and one or more antigen-specific cells.

12. (canceled)

13. A vaccine composition comprising a genetically modified PDC line according to claim 1 and a suitable vehicle for its administration.

14. The genetically modified PDC line according to claim 7, wherein the peptide antigens are derived from tumoral or viral proteins.

15. A method for in vitro amplification of antigen-specific cells, said method comprising co-culturing a genetically modified PDC line according to claim 6 with the antigen-specific cells.

16. The method according to claim 15, wherein the antigen-specific cells are mononuclear cells (MNC).

17. The method according to claim 15, wherein the antigen-specific cells are peripheral blood mononuclear cells (PBMC).

18. A method for preventing and/or treating cancers and/or infectious diseases in a patient in need thereof, comprising the injection in said patient of a PDC line genetically modified to express a cytokine selected from interleukin 15 (IL15) and interleukin 2 (IL2), said PDC line being irradiated and loaded with an antigen effective to promote an immune response for preventing and/or treating cancers and/or infectious diseases, wherein the antigen-specific cells of said patient and the cells of the genetically modified PDC line share at least one major histocompatibility complex (MHC) allele.

19. A method for preventing and/or treating cancers and/or infectious diseases in a patient in need thereof comprising incubating a PDC line genetically modified to express a cytokine selected from interleukin 15 (IL15) and interleukin 2 (IL2), said PDC line being irradiated and loaded with an antigen effective to promote an immune response for preventing and/or treating cancers and/or infectious diseases with antigen-specific cells of said patient to amplify the said antigen specific cells to amplify, and injecting the amplified antigen specific cells to said patient, wherein the cells of the genetically modified PDC line and the antigen-specific cells share at least one major histocompatibility complex (MHC) allele.
